(19) United States
(12) Patent Application Publication
(10) Pub. No.: US 2006/0068398 A1
(43) Pub. Date: Mar. 30, 2006

(54) Universal and target specific reagent beads for nucleic acid amplification

(75) Inventor: William A. McMillan, Cupertino, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Cepheid, Sunnyvale, CA (US)

(21) Appl. No.: 10/949,157

(22) Filed: Sep. 24, 2004

(51) Int. Cl. C12Q 1/68; C12P 19/34

(52) U.S. Cl. 435/6; 435/91.2

(57) ABSTRACT

The present invention relates to methods and compositions providing optimized reaction conditions for nucleic acid amplification reactions and for methods and compositions that reduce contamination of amplification reactions during set up.

### CROSS-REFERENCES TO RELATED APPLICATIONS

NOT APPLICABLE

### STATEMENT AS TO RIGHTS TO INVENTIONS MADE UNDER FEDERALLY SPONSORED RESEARCH OR DEVELOPMENT

NOT APPLICABLE

### REFERENCE TO A “SEQUENCE LISTING,” A TABLE, OR A COMPUTER PROGRAM LISTING APPENDIX SUBMITTED ON A COMPACT DISK

NOT APPLICABLE

### BACKGROUND OF THE INVENTION

In vitro nucleic acid amplification techniques provide powerful tools for detection and analysis of small amounts of nucleic acids. For example, the polymerase chain reaction (PCR) is a particularly well known and versatile thermocyclic method for the amplification of a nucleic acids (see e.g., PCR Technology: Principles and Applications for DNA Amplification Erlich, ed., (1992); PCR Protocols: A Guide to Methods and Applications, Innis et al., eds, (1990); R. K. Saiki, et al., Science 230:1350 (1985), and U.S. Pat. No. 4,683,202 to Mullis, et al.). PCR is easily adapted for high throughput screening, and can be used in numerous detection assays including; cloning DNA sequences, forensics, paternity testing, pathogen identification, and disease diagnosis, to list a few.

Despite the unquestioned utility of nucleic acid amplification reactions, artifacts frequently arise, often due to less than optimal reaction conditions. Factors that may influence the optimal reaction conditions for an amplification reaction include, but are not limited to, the amplification enzyme employed for the reaction, the ionic strength of the reaction buffer, the relative concentrations of amplification primers, or the concentration of template nucleic acids.

Optimization efforts, especially for mass production multiplex assays, can be tedious, labor intensive, and time-consuming. For example, the presence of more than one primer pair in the multiplex PCR increases the chance of forming spurious amplification products, such as primer dimers. Indeed, it is often impossible to predict the performance characteristics of a selected primer pair even among those that satisfy the general parameters of primer design. Furthermore, empirical testing and a trial-and-error approach is often the only means for testing several primer pairs, because the interaction of the primer pairs with each other can alter the performance of an individual primer pair over its performance when used alone in a reaction.

In addition to consideration of primer interactions, optimization of multiplex PCRs must also address difficulties involving poor sensitivity or specificity and/or preferential amplification of certain specific targets. Even after finding a reasonable set of primer concentrations, Mg²⁺ concentration, and amount of enzyme will need to be optimized. Therefore, because finding the right reaction conditions for an assay can be difficult, once optimal reaction conditions for a particular assay are established, it is important that they are easily and precisely reproducible.

Once optimization is achieved, reproducibility can sometimes prove difficult. This is especially the case where reaction components for multiple reactions must be mixed in a step-wise fashion for each assay. Adding and mixing trace amounts of each component in a separate manner provides multiple opportunities for cross contamination between samples and for contamination with minute amounts of unwanted extraneous nucleic acids from the environment. Thus, especially when numerous samples are to be analyzed, reliable, and accurate reproducibility of optimal reaction conditions may be difficult, time consuming, labor intensive, and will require skilled technicians.

Thus, there is a need in the art for compositions and methods that ensure reliable reproduction of optimal reaction conditions. In addition to providing reproducibly optimized reaction conditions, the compositions and methods would eliminate or reduce cross-over contamination between reactions, and would eliminate or reduce the introduction of unwanted exogenous nucleic acids and other contaminants into the reaction. The invention disclosed herein addresses these and other needs.

### SUMMARY

In one aspect, the invention provides a multiple bead assay system for nucleic acid amplification with an internal control oligonucleotide template, the multiple bead assay system comprising a set of reagent beads. The set of reagent beads includes a first lyophilized reagent bead comprising at least one enzyme for nucleic acid amplification, and further comprising nucleotides, buffers, salts, or cofactors. The set of reagent beads also includes a second lyophilized reagent bead comprising oligonucleotide primers for specific amplification of the control oligonucleotide template, and further comprising primers for specific amplification of at least one analyte nucleic acid sequence. In one embodiment the internal control oligonucleotide template is in the first lyophilized reagent bead, and in another embodiment the internal control oligonucleotide template is in the second lyophilized reagent bead. In some embodiments, the set of reagent beads further comprises a third lyophilized reagent bead, wherein the third lyophilized reagent bead comprises at least one probe for detection of nucleic acid amplification product. In some embodiments, the second lyophilized reagent bead further comprises probes for detecting the control oligonucleotide and the analyte nucleic acid sequence. In some embodiments, the second lyophilized reagent bead comprises primers for specific amplification of at least two or three analyte nucleic acid sequences, and the second lyophilized reagent bead further comprises probes for detecting the control oligonucleotide and the analyte nucleic acid sequences.

In another aspect, the invention provides a multiple bead assay system for nucleic acid amplification, the multiple bead assay system comprising a set of reagent beads. The set of reagent beads includes a first lyophilized reagent bead comprising at least one enzyme for nucleic acid amplification and further comprising nucleotides, buffers, salts, or cofactors. The set of reagent beads also includes a second lyophilized reagent bead comprising oligonucleotide primers for amplification of at least one analyte nucleic acid sequence. In one embodiment, the second lyophilized reagent bead comprises at least one probe for detection of nucleic acid amplification product. In another embodiment, the system further comprises a third lyophilized reagent bead containing at least one probe for detection of nucleic acid amplification product. In some embodiments, the second lyophilized reagent bead comprises primers for specific amplification of at least two or three analyte nucleic acid sequences, and the second lyophilized reagent bead further comprises probes for detecting the analyte nucleic acid sequences.

In another aspect, the invention provides a method for performing a nucleic acid amplification assay with an internal control oligonucleotide template. The method comprises the step of combining in aqueous solution (i) a first lyophilized reagent bead that comprises at least one enzyme for nucleic acid amplification, and nucleotides, buffers, salts, or cofactors and (ii) a second lyophilized reagent bead comprising oligonucleotide primers for specific amplification of the control oligonucleotide template and further comprising primers for specific amplification of at least one analyte nucleic acid sequence. The method further comprises the step of performing the nucleic acid amplification assay to amplify the internal control oligonucleotide and the at least one analyte nucleic acid sequence, if present. In one embodiment the internal control oligonucleotide template is in the first lyophilized reagent bead, and in another embodiment the internal control oligonucleotide template is in the second lyophilized reagent bead. In some embodiments, the method further includes the step of detecting the internal control oligonucleotide and the analyte nucleic acid sequence, e.g., by detecting a signal from a probe. In some embodiments, the second lyophilized reagent bead comprises probes for detecting the internal control oligonucleotide and the analyte nucleic acid sequence. In some embodiments, the method further comprises the step of combining a third lyophilized reagent bead in the aqueous solution, wherein the third lyophilized reagent bead comprises a probe for detection of nucleic acid amplification product. In some embodiments, the second lyophilized reagent bead comprises primers for amplification of at least two or three analyte nucleic acid sequences, and the step of performing the nucleic acid assay comprises amplifying the at least two or three analyte sequences, if present in the solution. In some embodiments, the second lyophilized reagent bead further comprises probes for detecting the internal control oligonucleotide and the analyte nucleic acid sequences, and the method further comprises the step of detecting the internal control oligonucleotide and the analyte nucleic acid sequences, if present.

Other aspects, objects and advantages of the present invention will be apparent from the detailed description that follows.

### DETAILED DESCRIPTION OF THE INVENTION

### Definitions

The phrase “multiple bead assay system” refers to an assay system wherein the components of the assay system are contained within more than one matrix, and each matrix has the form of a lyophilized bead.

A “bead”, as used herein, refers to a small, often round piece of material. A bead can have a spherical as well as a nearly spherical, e.g., elliptical, shape. In an exemplary embodiment, the beads have cross-sections which are between one millimeter and twenty-five millimeters.

The terms “nucleic acid amplification” or “amplification reaction” or “the amplification of a nucleic acid sequence”, refer to any chemical, including enzymatic, reaction that results in increased copies of a nucleic acid sequence. Amplification reactions include polymerase chain reaction (PCR) and ligase chain reaction (LCR) (see U.S. Pat. Nos. 4,683,195 and 4,683,202; Innis, et al., eds, PCR Protocols: A Guide to Methods and Applications (1990)), strand displacement amplification (SDA) (Walker, et al., Nucleic Acids Res. 20(7):1691-1696 (1992); Walker, PCR Methods Appl. 3(1):1-6 (1993)), transcription-mediated amplification (Phyffer, et al., J. Clin. Microbiol. 34:834-841 (1996); Vuorinen, et al., J. Clin. Microbiol. 33:1856-1859 (1995)), nucleic acid sequence-based amplification (NASBA) (Compton, Nature 350:91-92 (1991)), loop-mediated isothermal amplification (LAMP) (Notomi, et al., Nucleic Acids Res. 28:12 (2000)) and single primer amplification (SPA) (see U.S. Pat. Nos. 5,508,178, 5,595,891, and 5,612,199), rolling circle amplification (RCA) (Lisby, Mol. Biotechnol. 12(1):75-99 (1999)); Hatch, et al., Genet. Anal. 15(2):35-40 (1999)) and branched DNA signal amplification (bDNA) (see Iqbal, et al., Mol. Cell Probes 13(4):315-320 (1999)). Other amplification methods known to those of skill in the art include CPR (Cycling Probe Reaction), SSR (Self-Sustained Sequence Replication), QBR (Q-Beta Replicase), Re-AMP (formerly RAMP), RCR (Repair Chain Reaction), TAS (Transcription Based Amplification System), RT-PCR (Real Time PCR), and Reverse Transcriptase PCR.

The term “specific amplification” refers to amplification of a specified polynucleotide. In practical terms a nucleic acid may be specified for amplification by designing forward and reverse oligonucleotide primers that flank the nucleotide sequence to be amplified, or specification may be made by any other means known in the art.

The terms “nucleic acid” or “polynucleotide” or “nucleic acid sequences” refer to deoxyribonucleotides or ribonucleotides and polymers thereof in either single- or double-stranded form. The term encompasses nucleic acids containing known nucleotide analogs or modified backbone residues or linkages, which are synthetic, naturally occurring, or non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs). The term nucleic acid sequences encompasses sequences which are obtained or purified from natural sources, as well as sequences which are obtained or constructed from recombinant or synthetic chemical processes.

A “template” refers to a polynucleotide sequence that serves as a pattern for synthesis of another polynucleotide of complementary base sequence. In practical terms a template may comprise either a single or a double stranded polynucleotide sequence. A double stranded polynucleotide provides complementary single stranded polynucleotide sequences each of which may serve as a template for nucleotide synthesis in the reproduction and amplification of a double stranded molecule.

A “analyte nucleic acid sequence” refers to a single or double stranded polynucleotide sequence sought to be amplified in an amplification reaction.

A “probe” refers to a molecule that allows for the detecting of the polynucleotide sequence of interest. In certain embodiments, a probe comprises a polynucleotide sequence capable of hybridization to a polynucleotide sequence of interest. In other embodiments, a probe comprises an agent capable of intercalating into a polynucleotide sequence of interest. Examples of intercalating agents include ethidium bromide or SYBR Green. In other embodiments, the probe comprises a label. The probes are typically labeled either directly, as with isotopes, chromophores, lumiphores, chromogens, or indirectly, such as with biotin, to which a streptavidin complex may later bind. Thus, the labels of the present invention can be primary labels (where the label comprises an element that is detected directly or that produces a directly detectable element) or secondary labels (where the detected label binds to a primary label, e.g., as is common in immunological labeling). In some embodiments, labeled nucleic acid probes are used to detect hybridization. Nucleic acid probes may be labeled by any one of several methods typically used to detect the presence of hybridized polynucleotides. In some embodiments, label detection occurs through the use of autoradiography with ³H, ¹²⁵I, ³⁵S, ¹⁴C, or ³²P-labeled probes or the like. Other labels include, e.g., ligands which bind to labeled antibodies, fluorophores, chemiluminescent agents, intercalating agents, enzymes, and antibodies which can serve as specific binding pair members for a labeled ligand. An introduction to labels, labeling procedures, and detection of labels is found in Polak and Van Noorden Introduction to Immunocytochemistry, 2nd ed., Springer Verlag, NY (1997); and in Haugland Handbook of Fluorescent Probes and Research Chemicals, a combined handbook and catalogue Published by Molecular Probes, Inc. (1996).

A “signal from a probe” or a “probe signal” refers to any property or parameter that serves to convey information regarding that element of an assay or reaction that is detected by the probe. The signal from the probe can be detected by any means known in the art. For example, signal can be detected through the use of autoradiography when ¹²⁵I, ³⁵S, ¹⁴C, or ³²P-labeled probes are employed. Other means of detecting a signal from a probe may include, but are not limited to detection of a chromophore or lumiphore, or may be detected with various immunological assay as is known in the art.

The term “internal control”, as used herein, refers to a control reaction run in parallel, in the same container, and under the same conditions as a reaction of interest, that functions as a standard of comparison. The term “internal control oligonucleotide” refers to a template nucleic acid sequence whose amplification functions as a control reaction for a nucleic acid amplification assay.

An “amplification reagent” or “reagent for polynucleotide amplification”, as used herein, refers to a reagent for use to amplify nucleic acids in an amplification reaction. The reagent can, but need not, comprise all of the components required for an amplification reaction. Examples of components of an amplification reaction can include, but are not limited to: nucleic acids, including templates, primers or deoxynucleotide triphosphates, a DNA polymerase (e.g., Taq polymerase, polymerase complexed with a hot start antibody such as Platinum polymerase), buffers (e.g., Tris (2-Amino-2-hydroxymethyl-1,3-propanediol), HEPES (N-(2-Hydroxyethyl)piperazine-N′-(2-ethanesulfonic acid)), etc.), salts such as magnesium and/or potassium-based salts, disaccharides or disaccharide derivatives, carrier proteins, detergents, DMSO, or other like agents.

The term “oligonucleotide primer” refers to a nucleic acid segment that is complementary to a target nucleic acid sequence that is subject to amplification in an amplification reaction. An “oligonucleotide primer” may be designed as a “forward polynucleotide primer”, or “5′ primer”, meaning that it is complementary to and hybridizes with the 5′ end of the target nucleic acid sequence subject to amplification. Alternatively, an “oligonucleotide primer” may be a “reverse polynucleotide primer”, or “3′ primer”, meaning that it is complementary to and hybridizes with the 3′ end of the target nucleic acid sequence subject to amplification.

I. Introduction

Establishing optimal conditions for multiplex assays can be a tedious, time-consuming, and expensive process. Careful attention must be given to adjusting primer concentrations, Mg²⁺ concentration, amount of enzyme, and etc. In many cases, the results are disappointing and further extensive optimization may be required. Therefore, there is a need in the art for compositions and methods that can be used effectively to minimize the negative consequences of poorly optimized amplification reactions, and that can reduce costs of repeated optimization. Thus, in one aspect the invention provides a multiple bead assay system for nucleic acid amplification with an internal control oligonucleotide template, comprising a set of reagent beads. The set of reagent beads is comprised of a first lyophilized reagent bead comprising at least one enzyme for nucleic acid amplification, and nucleotides, buffers, salts, or cofactors, and a second lyophilized reagent bead comprising oligonucleotide primers for specific amplification of the control oligonucleotide template, and primers for specific amplification of at least one analyte nucleic acid sequence.

In another aspect, the invention provides a method for performing a nucleic acid amplification assay with an internal control oligonucleotide template comprising the steps of: (i) combining in aqueous solution a first lyophilized reagent bead that comprises at least one enzyme for nucleic acid amplification, and nucleotides, buffers, salts, or cofactors and a second lyophilized reagent bead comprising oligonucleotide primers for specific amplification of the control oligonucleotide template, and primers for specific amplification of at least one analyte nucleic acid sequence, and (ii) allowing the reaction to perform.

II. Nucleic Acid Amplification Reactions

The invention can be used to enhance the performance of any amplification reaction. Amplification reactions take many forms, depending on the nature of the molecule being amplified and on the context in which it occurs. For example amplification reactions may comprise reactions such as polymerase chain reaction (PCR, U.S. Pat. Nos. 4,683,195; 4,683,202; and 4,965,188), nucleic acid sequence based amplification (NASBA, U.S. Pat. Nos. 5,409,818; 5,130,238; and 5,554,517), transcription-mediated amplification (TMA, U.S. Pat. No. 5,437,990), self-sustained sequence replication (3SR, Fahy, et al., PCR Methods & Appl. 1: 25-33, 1991), ligation chain reaction (LCR, U.S. Pat. Nos. 5,494,810 and 5,830,711), continuous amplification reaction or (CAR, U.S. Pat. No. 6,027,897), linked linear amplification of nucleic acids (LLA, U.S. Pat. No. 6,027,923) and strand displacement amplification (SDA, U.S. Pat. Nos. 5,455,166; 5,712,124; 5,648,211; 5,631,147), and methods to increase a signal produced in the presence of a polynucleotide, such as rolling circle amplification (RCA, U.S. Pat. No. 5,854,033), cycling probe reaction (CPR, U.S. Pat. Nos. 4,876,187 and 5,011,769 and 5,660,988), branched chain amplification (U.S. Pat. Nos. 4,775,619 and 5,118,605 and 5,380,833 and 5,629,153) among others. This multitude of methods may be conveniently divided two groups depending on whether the temperature during the reaction is cycled between heating and cooling steps (thermocyclic reactions), or maintained at a constant temperature (isothermic reactions).

Thermocyclic Amplification Reactions

Amplification of an RNA or DNA template using thermocyclic reactions is well known (see e.g. U.S. Pat. Nos. 4,683,195 and 4,683,202; PCR Protocols: A Guide to Methods and Applications Innis et al., eds, 1990, each of which is herein incorporated by reference). Methods such as polymerase chain reaction (PCR) can be used to amplify nucleic acid sequences of target DNA sequences directly from mRNA, from cDNA, from genomic libraries or cDNA libraries. Exemplary PCR reaction conditions typically comprise either two or three step cycles, wherein two step cycles have a denaturation step followed by a hybridization/elongation step, and three step cycles comprise a denaturation step followed by a hybridization step followed by a separate elongation step.

Thermocyclic nucleic acid amplification technologies such as polymerase chain reaction (PCR), and ligase chain reaction (LCR) are well known.

Isothermic Amplification reactions

Isothermic amplification reactions are also known and can be practiced according to the methods of the invention. Examples of isothermic amplification reactions include strand displacement amplification (SDA) (Walker, et al. Nucleic Acids Res. 20(7):1691-6 (1992); Walker PCR Methods Appl 3(1): 1-6 (1993)), transcription-mediated amplification (Phyffer, et al., J. Clin. Microbiol. 34:834-841 (1996); Vuorinen, et al., J. Clin. Microbiol. 33:1856-1859 (1995)), nucleic acid sequence-based amplification (NASBA) (Compton, Nature 350(6313):91-2 (1991), rolling circle amplification (RCA) (Lisby, Mol. Biotechnol. 12(1):75-99 (1999)); Hatch et al., Genet. Anal. 15(2):35-40 (1999)) and branched DNA signal amplification (bDNA) (see, e.g., Iqbal et al., Mol. Cell Probes 13(4):315-320 (1999)). Other amplification methods known to those of skill in the art include CPR (Cycling Probe Reaction), SSR (Self-Sustained Sequence Replication), SDA (Strand Displacement Amplification), QBR (Q-Beta Replicase), Re-AMP (formerly RAMP), RCR (Repair Chain Reaction), TAS (Transcription Based Amplification System), and HCS.

Multiplex Reactions

The methods of the invention can also be practiced in traditional multiplex reactions. Multiplex PCR results in the amplification of multiple polynucleotide fragments in the same reaction (see, e.g., PCR PRIMER, A LABORATORY MANUAL, Dieffenbach, ed. 1995 Cold Spring Harbor Press, pages 157-171, which is herein incorporated by reference). In multiplex PCR, multiple, different target templates can be added and amplified in parallel in the same reaction vessel. Multiplex PCR assays are well known in the art. For example, U.S. Pat. No. 5,582,989 discloses the simultaneous detection of multiple known DNA sequence deletions.

Real-Time Reporters for Multiplex PCR

The methods of the invention can be practiced in the execution of real time PCR, or “TaqMan” assays. Real time PCR is known in the art. In this embodiment of the invention, the lyophilized reagent beads comprise a probe that binds to a target template. As is known in the art, TaqMan probes contain two dyes, a reporter dye (e.g. 6-FAM) at the 5′ end and a quencher dye (e.g. Black Hole Quencher) at the 3′ end. During the reaction, the 5′ to 3′ nucleolytic activity of the Taq polymerase enzyme cleaves the probe between the reporter and the quencher thus resulting in increased fluorescence of the reporter. Accumulation of PCR products is detected directly by monitoring the increase in fluorescence of the reporter dye.

Quantitation of Amplification Reactions

Accumulation of amplified product can be quantified by any method known to those in the art. For instance, the standard curve method may be used to determine relative or absolute quantitation of amplification products. In other embodiments, amplification reactions can be quantified directly by blotting them onto a solid support and hybridizing with a radioactive nucleic acid probe.

Reaction Components

Primers and Oligonucleotide Probes

The principles of primer design are well known to those of skill in the art, and are described in a number of references, e.g., Ausubel et al., supra; and PCR Protocols: A Guide to Methods and Applications, Innis et al., eds., 1990, Rychlik, W., Selection of Primers for Polymerase Chain Reaction in B A White, ed., Methods in Molecular Biology, Vol. 15: PCR Protocols: Current Methods and Applications, (1993), pp 31-40, Humana Press, Totowa N.J., and Rychlik et al., Nucleic Acids Research, 18, (12): 6409-6412, and Breslauer et al., Proc. Natl. Acad. Sci. USA, 83: 3746-3750, each of which is herein incorporated by reference. Special primer design considerations for specific non-PCR amplification reactions can also be found, for example, in the following references: strand displacement amplification (SDA) Walker, et al. Nucleic Acids Res. 20(7):1691-6 (1992); Walker PCR Methods Appl 3(1):1-6 (1993)), transcription-mediated amplification (Phyffer, et al., J. Clin. Microbiol. 34:834-841 (1996); Vuorinen, et al., J. Clin. Microbiol. 33:1856-1859 (1995), nucleic acid sequence-based amplification (NASBA) Compton, Nature 350 (6313):91-2 (1991), rolling circle amplification (RCA) Lisby, Mol. Biotechnol. 12(1):75-99 (1999); Hatch et al., Genet. Anal. 15(2):35-40 (1999) and branched DNA signal amplification (bDNA) Iqbal et al., Mol. Cell Probes 13(4):315-320 (1999).

In general, primers that have melting temperatures in the range of 50° C. to about 75° C. are preferred. As is practiced by those skilled in the art, the formula Tₘ=[2(A+T)]+[4(G+C)] can be used to calculate the predicted melting temperature of the primers. Alternatively, commercially available primer design software can be used to more accurately calculate melting temperature, especially when the primers are greater then about 25 nucleotides in length. Primer sequences are frequently selected to have 50-60% G and C composition, which for a 20 mer oligonucleotide, implies a melting temperature in the range of 60° C.-68° C. However, the final composition of the primer for the control non-natural nucleic acid sequence will be such that the G−C content allows the control primer to have a melting temperature that matches that of the primer(s) for amplification of the analyte nucleic acid sequence(s).

The oligonucleotide primers of the invention may be conveniently synthesized on an automated DNA synthesizer, e.g., an Applied Biosystems, Inc. (Foster City, Calif.) model 392 or 394 DNA/RNA Synthesizer, using standard chemistries, such as phosphoramidite chemistry, e.g., disclosed in the following references: Beaucage and Lyer, Tetrahedron, 48: 2223-2311 (1992); Molko et al., U.S. Pat. No. 4,980,460; Koster et al., U.S. Pat. No. 4,725,677; Caruthers et al., U.S. Pat. Nos. 4,415,732; 4,458,066; and 4,973,679; and the like. Alternative chemistries, e.g., resulting in non-natural backbone groups, such as phosphorothioate, phosphoramidate, and the like, may also be employed provided that the hybridization efficiencies of the resulting oligonucleotides and/or cleavage efficiency of the 5′ to 3′ nuclease activity of the polymerase employed are not adversely affected. The primers can be labeled with radioisotopes, chemiluminescent moieties, or fluorescent moieties.

Analyte Nucleic Acid Sequences

Analyte nucleic acid sequences may be double or single-stranded DNA or RNA from any biological source, e.g., a bacterium, an animal, a plant, etc. Analyte nucleic acid sequences may be isolated using a variety of techniques. For example, methods are known for lysing organisms and preparing extracts or purifying DNA (see, e.g. Ausubel et al., eds., 1994-1998, Current Protocols in Molecular Biology Volumes 1-3, John Wiley & Sons, Inc.) Alternatively, total RNA or polyA+ RNA can be reverse transcribed to produce cDNA that can serve as the target DNA.

Generally, the nomenclature and the laboratory procedures in recombinant DNA technology described below are those well known and commonly employed in the art. Standard techniques are used for cloning, DNA and RNA isolation, amplification and purification. Generally enzymatic reactions involving DNA ligase, DNA polymerase, restriction endonucleases and the like are performed according to the manufacturer's specifications. These techniques and various other techniques are generally performed according to Sambrook et al., Molecular Cloning—A Laboratory Manual, Cold Spring Harbor Laboratory, Cold Spring Harbor, N.Y., (1989) or Ausubel et al., supra.

Synthetic oligonucleotides can be used to construct recombinant nucleic acids for use as probes, primers, or internal control oligonucleotides. Oligonucleotides can be chemically synthesized using an automated DNA synthesizer as described above in the “Primers and oligonucleotide probes” section.

Buffers

Buffers that may be employed are borate, phosphate, carbonate, barbital, Tris, etc. based buffers. See Rose et al., U.S. Pat. No. 5,508,178. The pH of the reaction should be maintained in the range of about 4.5 to about 9.5. See U.S. Pat. No. 5,508,178. The standard buffer used in amplification reactions is a Tris based buffer between 10 and 50 mM with a pH of around 8.3 to 8.8. See Innis et al., supra.

One of skill in the art will recognize that buffer conditions should be designed to allow for the function of all reactions of interest. Thus, buffer conditions can be designed to support the amplification reaction as well as any enzymatic reactions associated with producing signals from probes. A particular reaction buffer can be tested for its ability to support various reactions by testing the reactions both individually and in combination.

Salt Concentration

The concentration of salt present in the reaction can affect the ability of primers to anneal to the target nucleic acid. See Innis et al. Potassium chloride is added up to a concentration of about 50 mM to the reaction mixture to promote primer annealing. Sodium chloride can also be added to promote primer annealing. See Innis et al.

Magnesium Ion Concentration

The concentration of magnesium ion in the reaction can be critical to amplifying the desired sequence(s). See Innis et al. Primer annealing, strand denaturation, amplification specificity, primer-dimer formation, and enzyme activity are all examples of parameters that are affected by magnesium concentration. See Innis et al. Amplification reactions should contain about a 0.5 to 2.5 mM magnesium concentration excess over the concentration of dNTPs. The presence of magnesium chelators in the reaction can affect the optimal magnesium concentration. A series of amplification reactions can be carried out over a range of magnesium concentrations to determine the optimal magnesium concentration. The optimal magnesium concentration can vary depending on the nature of the target nucleic acid(s) and the primers being used, among other parameters.

Deoxynucleotide Triphosphate Concentration

Deoxynucleotide triphosphates (dNTPs) is added to the reaction to a final concentration of about 20 μM to about 300 μM. Each of the four dNTPs (G, A, C, T) should be present at equivalent concentrations. See Innis et al.

Nucleic Acid Polymerase

A variety of DNA dependent polymerases are commercially available that will function using the methods and compositions of the present invention. Indeed, any polymerase known in the art can be used. As an example, Taq DNA Polymerase may be used to amplify target DNA sequences. The PCR assay may be carried out using as an enzyme component a source of thermostable DNA polymerase suitably comprising Taq DNA polymerase which may be the native enzyme purified from Thermus aquaticus and/or a genetically engineered form of the enzyme. Other commercially available polymerase enzymes include, e.g., Taq polymerases marketed by Promega or Pharmacia. Other examples of thermostable DNA polymerases that could be used in the invention include DNA polymerases obtained from, e.g., Thermus and Pyrococcus species. Concentration ranges of the polymerase may range from 1-5 units per reaction mixture. The reaction mixture may typically be between 20 and 100 μl.

In some embodiments, a “hot start” polymerase can be used to prevent extension of mispriming events as the temperature of a reaction initially increases. Hot starts are particularly useful in the context of multiplex PCR. Hot start polymerases can have, for example, heat labile adducts requiring a heat activation step (typically 95° C. for approximately 10-15 minutes) or can have an antibody associated with the polymerase to prevent activation.

Other Agents

Assorted other agents are sometime added to the reaction to achieve the desired results. For example, DMSO can be added to the reaction, but is reported to inhibit the activity of Taq DNA Polymerase. Nevertheless, DMSO has been recommended for the amplification of multiple target sequences in the same reaction. See Innis et al. Stabilizing agents such as gelatin, bovine serum albumin, and non-ionic detergents (e.g. Tween-20) are commonly added to amplification reactions. See Innis et al.

III. Optimization of Amplification Reactions

Optimization of reaction conditions is essential for successful amplification reactions. Optimization is however, often an empirical process and can therefore be time consuming and labor intensive. However, initial optimization efforts focused on achieving efficient denaturation of the target, and improving the rate at which primers anneal to their target and the rate at which annealed primers are extended along the desired sequence during the early, middle, and late cycles of the amplification often lead to successful reaction optimization.

Factors preventing optimal annealing rates include poorly designed primers and suboptimal buffer constituents and annealing temperature. The extension rate of specific primer-target hybrids depends on the activity of the enzyme, availability of essential components such as deoxyribonucleoside triphosphates (dNTPs), and the nature of the target DNA. Thus, there are a number of modifications that may be the focus of optimization efforts to improve PCR performance.

Primer Design

Although ideally all the primer pairs in a multiplex PCR should enable similar amplification efficiencies for their respective target, in multiplex PCR, the annealing efficiencies of the different primers in the reaction are usually dissimilar. Because of the various primer-primer, and primer-template interactions, the Tm of each primer does not necessarily provide a good indication of its annealing efficiency in a multiplex reaction. Nonetheless, optimization may begin with attention to primer design parameters such as homology of primers with their target nucleic acid sequences, their length, the GC content, and their concentration. Primers should have nearly identical optimum annealing temperatures with a primer length of 18 to 30 bp or more and a GC content of 35 to 60%, and should not display significant homology either internally or to one another. Other factors such as the 3′-end sequence of primers may affect the efficiency of primer extension by Taq DNA polymerase and so should be carefully considered as part of the optimization process.

Primer Concentration

Total primer concentration may also strongly influence the outcome of multiplex amplification. Typically, a primer concentration of 0.2-0.5 μM is used in conventional PCR. In contrast, the total primer concentration in multiplex PCR can be as high as 2-4 μM, depending on the number of different primer pairs in the reaction. The large number of primers often results in the generation of nonspecific PCR products and primer-dimers, reducing the specificity and sensitivity of the multiplex PCR. Using a stringent hot start to increase PCR specificity can prevent the generation of these nonspecific products.

Additives and Buffers

PCR additives, such as dimethyl sulfoxide, glycerol, bovine serum albumin, or betaine, may be of benefit in multiplex PCRs. The components may act to prevent the stalling of DNA polymerization, which can occur through the formation of secondary structures within regions of template DNA during the extension process. Such cosolvents may also act as destabilizing agents, reducing the melting temperature of GC-rich sequences, or as osmoprotectants, increasing the resistance of the polymerase to denaturation

In addition, by providing a balanced combination of salts nonspecific primer binding may be avoided. For example, optimized NH⁴⁺ concentration can dramatically improve multiplex amplification results. NH⁴⁺, which exists predominantly as ammonia (NH₃) under thermal-cycling conditions, interacts with the relatively weak hydrogen bonds formed when primers bind nonspecifically to the template DNA and destabilizes these nonspecifically bound primers. Therefore, NH⁴⁺ concentration can play a major role in balancing the reaction to favor specific and efficient binding of primers and template.

Enzymes

Finally enzyme type and concentration may affect the outcome of a multiplex amplification reaction. For example, in some cases of multiplex PCR, a Taq DNA polymerase concentration four to five times greater than that required in uniplex PCR may be necessary to achieve optimal nucleic acid amplification.

### CONCLUSION

Those skilled in the art may wish to refer to Innis et al., “Optimization of PCR's”, PCR Protocols: A Guide to Methods and Applications Academic Press, 1990, or Ausubel et al. (supra), for further information regarding techniques that can be used successfully to influence the specificity, fidelity and yield of the desired PCR products.

IV Lyophilized Beads

A “lyophilized bead” comprises an excipient and a biological reagent. The beads are produced by forming a bead buffer formulation (containing the excipient and biological reagent), creating the beads from the bead buffer formulation, and finally freeze-drying the beads. The produced bead can possess a variety of morphologies and shapes. Exemplary shapes include spherical, near spherical, elliptical or round structures. Exemplary morphologies include smooth or slightly roughened surfaces.

Excipient

Excipients are more or less inert substances added to a material in order to confer a suitable consistency or form to the material. A large number of excipients are known to those of skill in the art and can comprise a number of different chemical structures. Examples of excipients, which may be used in the present invention, include carbohydrates, such as sucrose, glucose, trehalose, melezitose, dextran, and mannitol; proteins such as BSA, gelatin, and collagen; and polymers such as PEG and polyvinyl pyrrolidone (PVP). The total amount of excipient in the lyophilized bead may comprise either single or multiple compounds.

Excipients are added to reagent formulations for a variety of reasons, and each excipient has its own advantages and disadvantages. Thus, usually more than one excipient is required in the formulation to provide all the desirable attributes. For example, an excipient may be added to a formulation to be freeze dried so as to reduce the time for reconstitution. (see, e.g., Carpenter and Crowe, “The Mechanism of Cryoprotection of Proteins by Solutes,” Cryobiology, 25: 244-255 (1988)). Alternatively, excipients may be added to the formulation to facilitate attainment of the shape of the final lyophilized product. For example, excipient can be added to facilitate or prevent the product from attaining a bead like shape.

The type of excipient may also be a factor in controlling the amount of bead hygroscopy. Lowering bead hygroscopy can enhance the bead's integrity (accuracy of weighing beads) and cryoprotectant abilities. However, removing all water from the bead would have deleterious effects on those reaction components, proteins for example, that require certain amounts of bound water in order to maintain proper conformations. In general, the excipient level in the beads should be adjusted to allow moisture levels of less than 3%.

Naturally, there are limits to the amount of excipient which can be added to form a bead. If the amount of excipient is too low, the material does not coalesce to form a bead-like shape. At the high end, excipient amounts are limited by the solubility of the excipient in the bead buffer formulation. The amount is also dependent upon the properties of the excipient. In an exemplary embodiment, trehalose is present from between 5% to 20% (w/v). In another exemplary embodiment, mannitol is present from between 2% to 20% (w/v). In yet another exemplary embodiment, mannitol is present from between 2% to 20% (w/v) and dextran is present from between 0.5% to 5% (w/v). In still another exemplary embodiment, mannitol is present in the lyophilized bead in a weight percentage of between 40% to 75% (w/w).

Preparation of Reagent Beads

Bead Formation

The reagent spheres of the present invention are prepared from reagents suitable for any of the protein based analytical assays of the invention. Typically, an aqueous solution comprising the reagents is prepared. To ensure uniform composition of the reagent spheres, the solution is made homogeneous and all constituents are fully dissolved or in suspension. The final volume per drop of the reagent emulsion is often small, between 2-20 μL, to allow a working volume of 5-200 μL when the lyophilized bead is dissolved in a working solution.

The drops are uniform and precisely measured so that the resulting dried reagent spheres have uniform mass. Using a volumetric or gravimetric dispensing system such as those made by FMI or IVEC has been shown to work well. A time/pressure method such as that used to dispense adhesives also works well.

When the drops are uniform and precisely measured, the imprecision of the mass (coefficient of weight variation) of the reagent spheres prepared from the drops is less than about 3%, and preferably between about 0.3% and about 2.5%. To further decrease the weight variation, the aqueous solution may be degassed using a vacuum pump or vacuum line before the drops of solution are dispensed.

Individual drops of the solution are formed into beads either by dropping the dispensed emulsion onto a cryogenic liquid or onto a cryogenically cooled solid surface, or alternatively, by first dispensing the emulsion a drying surface that facilitates bead formation before the bead is frozen. The composition and shape of such a drying surface determines the drop shape as well as the ease of release from the surface after drying. In some embodiments, the dispensed emulsion is placed upon glass, polystyrene, wax paper, Delrin, or a coated aluminum pan (coatings can be nickel, Teflon, titanium nitrite and combinations thereof).

Bead formation can also occur by dropping the dispensed emulsion onto a cryogenic liquid or onto a cryogenically cooled solid surface. Cryogenic is defined as a liquefied or solidified gas having a normal boiling or sublimation point below about −75° C.; in some cases, this point is below about −150° C. In an exemplary embodiment, the cryogenic material is nitrogen, Freon, or carbon dioxide. The frozen beads are recovered and then freeze dried to a moisture content of less than about 10%. In some cases, the moisture content is less than 3%.

Bead Lyophilization

Lyophillization is extremely useful for enhancing the shelf life and stability o biologicals that are thermolabile and/or unstable in aqueous solution. Vacuum drying, desiccant drying, and freeze-drying of the biological reagent droplets can be utilized for drying the bead material. A standard freeze-drier (such as a VirTis GENESIS) with a control modified to allow operation at partial vacuums is sufficient.

As noted above, the product to be made using lyophilization is prepared as an aqueous solution or suspension, formed into drops then cooled rapidly to a predetermined temperature that often approaches −50° C. The frozen masses are then lyophilized by methods known in the art, to produce the reagent spheres. The freezing chamber is sealed and the frozen material subjected to heat under high vacuum conditions. The liquid portion sublimes, leaving the desired solid material.

Typically, the frozen drops are lyophilized for about 4 hours to about 24 hours at about 50 to about 450 mTorr, preferably, about 6 hours at about 200 mTorr. The final reagent spheres typically comprise less than about 6% residual moisture, preferably less than about 3%. Reabsorbtion of moisture can occur after lyophilization, necessitating quick removal from the chamber to conditions of low humidity environment. The dried material is porous upon sublimation of ice crystals. This surface character influences the rate of moisture reabsorbtion, dissolution in solution, and shelf life of the dried product.

All publications and patent applications cited in this specification are herein incorporated by reference as if each individual publication or patent application were specifically and individually indicated to be incorporated by reference.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

### EXAMPLES

The following examples are provided by way of illustration only and not by way of limitation. Those of skill will readily recognize a variety of noncritical parameters which could be changed or modified to yield essentially similar results.

### Example 1

Making Reagent Beads

Two sets of lyophilization buffers were mixed for the preparation of the reagent beads. The first set of lyophilization buffers employs separate buffers for the enzyme (universal bead) and for the assay specific reagent bead. The buffers are distinguished by the pH and the molarity of the buffering agent.

The second lyophilization buffer set is a single buffer formulated for use with both the enzyme (universal bead) and with the assay specific reagent bead.

Table 1 provides the formulation for the lyophilization buffer used to prepare the universal reagent bead for the enzyme reagent. Table 2 provides the formulation for the lyophilization buffer used to prepare the bead comprising the assay specific reagents.

TABLE 1
Lyophilization Buffer for Enzyme Reagent pH 7.15 Formulation
To this formulation the appropriate components are added

| | | | | 4 × Lyophilization |
|---|---|---|---|---|
| | | 4 × Lyophilization | | Concentration |
| Component | Vendor/Part # | Concentration | | (gm/100 mL) |
| HEPES Salt (MW = 260.3) | Sigma H3784 | 17.5 | mM | 0.456 |
| HEPES Acid (MW = 238.3) | Sigma H4034 | 14.5 | mM | 0.346 |
| KCl (FW = 74.55) | Sigma P9541 | 60.0 | mM | 0.447 |
| MgCl2 (FW = 95.21)* | Sigma M8266 | 24.0 | mM | 0.229 |
| BSA | Sigma A7638 | 0.18-0.36% | w/v | 0.18-0.36 |
| MIT | Sigma M6045 | 0.1% | w/v | 0.10 |
| Mannitol | Sigma M9546 | 11.0% | w/v | 11.0 |
| Dextran T-40 | AmershemPharmacia | 2.5% | w/v | 2.50 |
| Tween 20. | Pierce #28320 | 0.2% | v/v | 2.0 mL of 10% |
| | | | | stock |
| Antifoam SE-15. | Sigma A8582 | 0.024% | v/v | 0.24 mL of 10% |
| | | | | stock |
| (pH 7.15 ± 0.1) | | | | |

*MgCl2 concentration can be optimized for a specific assay

TABLE 2
Lyophilization Buffer for target specific reagent pH 8.35 Formulation
To this formulation the appropriate components are added

| | | | | 4 × Lyophilization |
|---|---|---|---|---|
| | | 4 × Lyophilization | | Concentration |
| Component | Vendor/Part # | Concentration | | (gm/100 mL) |
| HEPES Salt (MW = 260.3) | Sigma H3784 | 117.0 | mM | 3.046 |
| HEPES Acid (MW = 238.3) | Sigma H4034 | 8.0 | mM | 0.191 |
| KCl (FW = 74.55) | Sigma P9541 | 60.0 | mM | 0.447 |
| MgCl2 (FW = 95.21)* | Sigma M8266 | 24.0 | mM | 0.229 |
| BSA | Sigma A7638 | 0.18-0.36% | w/v | 0.18-0.36 |
| MIT | Sigma M6045 | 0.1% | w/v | 0.10 |
| Mannitol | Sigma M9546 | 11.0% | w/v | 11.0 |
| Dextran T-40 | Amersham Pharmacia | 2.5% | w/v | 2.50 |
| Tween 20. | Pierce #28320 | 0.2% | v/v | 2.0 mL of 10% |
| | | | | stock |
| Antifoam SE-15. | Sigma A8582 | 0.024% | v/v | 0.24 mL of 10% |
| | | | | stock |
| (pH 8.35 ± 0.1) | | | | |

*MgCl2 concentration can be optimized for the specific assay

The lyophilization buffer for the enzyme reagent will contain Taq polymerase enzyme and dNTP's. The lyophilization buffer for the assay specific reagent will contain the primers, fluorescent probes, internal control DNA, and other necessary components.

The reaction pH is controlled by the buffering capacity of the assay specific reagent (ASR) buffer. The HEPES buffer concentration is much higher in the assay specific reagent (125.0 mM) than in the universal enzyme buffer (32.0 mM). When the HEPES buffer from assay specific reagent and the enzyme reagents are mixed together a final PCR reaction pH of 8.00 is obtained, which is favorable for the PCR reaction.

In order to compare the properties of reagents stabilized in different lyophilization formulas, a universal lyophilization buffer formulation was prepared so that both the enzyme and the assay specific reagent (ASR) could be formulated into beads starting with a pH 8.00 buffer.

Formulation of the universal lyophilization buffer formulation is shown in Table 3. The formulation comprises 100 mM HEPES, pH 8.00±0.1. The appropriate active components are added to this buffer formulation in preparing the enzyme and assay specific reagents.

TABLE 3
Lyophilization Buffer for Enzyme and ASR pH 8.00 Formulation
To this formulation the appropriate components are added

| | | | | 4 × Lyophilization |
|---|---|---|---|---|
| | | 4 × Lyophilization | | Concentration |
| Component | Vendor/Part # | Concentration | | (gm/100 mL) |
| HEPES Salt (MW = 260.3) | Sigma H3784 | 83.0 | mM | 2.16 |
| HEPES Acid (MW = 238.3) | Sigma H4034 | 17.0 | mM | 0.405 |
| KCl (FW = 74.55) | Sigma P9541 | 60.0 | mM | 0.447 |
| MgCl2 (FW = 95.21)* | Sigma M8266 | 24.0 | mM | 0.229 |
| BSA | Sigma A7638 | 0.18-36% | w/v | 0.18-36 |
| MIT | Sigma M6045 | 0.1% | w/v | 0.10 |
| Mannitol | Sigma M9546 | 11.0% | w/v | 11.0 |
| Dextran T-40 | Amershem Pharmacia | 2.5% | w/v | 2.50 |
| Tween 20. | Pierce #28320 | 0.2% | v/v | 2.0 mL of 10% |
| | | | | stock |
| Antifoam SE-15. | Sigma A8582 | 0.024% | v/v | 0.24 mL of 10% |
| | | | | stock |
| (pH 8.00 ± 0.1) | | | | |

*MgCl2 concentration can be optimized for the specific assay

All the lyophilization buffers mentioned above are a 4× concentrate. A 100 μL final reaction volume requires 12.5 uL of enzyme reagent (which contains lyophilization buffer, enzyme, and dNTP's) and 12.5 uL of assay specific reagent (which contains lyophilization buffer, primers, probes, and internal control DNA, etc.) and 75 μL water containing plus the sample.

In preparing the lyophilized beads the lyophilization buffer is prepared at only 72% of its final volume in order to compensate for volume displacement which will occur as a result of other liquid components are added later. Addition of other components such as dNTP's to the enzyme reagent, and primers and probes for the assay specific reagent dictate the final volume required to give the desired bead size.

### Example 2

Carrying Out PCR Assay With Reagent Beads

Assay Protocols:

All the assays were run on Cepheid Smart Cycler®, Cepheid Inc., Sunnyvale, Calif. using software v2.0c: S/N 200019, 200016, 900039, 900339, and 900211
Computers S/N: 8BDW021, 23WSG31
Ba Lysed spores or DNA
Enzyme; Ampli Taq lot #E01902 (Roche)+ hot start antibody TAKARA (lot #N1803-1)
Cepheid Assay specific primers and fluorescent probes
   Procedures
Six replicates for each sample containing 0 (negative control), 0.1 pg, 1.0 pg, 10.0 pg, Ba DNA/25 μL reaction was assayed for the simplex and duplex assays.
Simplex assays comprise only one template-primer-probe set, and duplex assays comprise two primer and probe sets.

And six replicates of samples containing 0(Negative control), 4×10², 4×10³, 4×10⁴ lysed Ba spores per 85 uL reaction were assayed for the 4-Plex assay.

ASSAY PROTOCOL ON SMART CYCLER ® SOFTWARE V2.0C

| | | |
|---|---|---|
| Step 1 | 95° C., 30 seconds | Optics off |
| Step 2 | 95° C., 1 second | Optics off |
| 45 cycles | 65° C., 20 seconds | Optics on |

For each reaction the cycle threshold (Ct), and the end point fluorescence (EP) were measured. The cycle threshold (Ct), correlates with the log-linear phase of PCR amplification and is the first cycle in which there is significant increase in fluorescence above the background.

TABLE 6
Ba Simplex and Duplex Assays
Cycle Threshold and End point fluorescence with target DNA

| DNA concentrate | Ba Simplex Assay | | Ba Duplex Assay | |
|---|---|---|---|---|
| on (pg/25 uL | Cycle | End Point | Cycle | End Point |
| reaction) | Threshold | Flouresence | Threshold | Flouresence |
| 0.0 | 0.0 | −0.2 | 0.0 | −6.8 |
| 0.1 | 32.7 | 343.2 | 32.3 | 362.0 |
| 1.0 | 29.2 | 405.2 | 28.9 | 433.4 |
| 10.0 | 25.8 | 421.6 | 25.4 | 509.2 |

Each value represents an average of six replicates

TABLE 7
Ba 4-Plex Assays
Cycle Threshold and End point fluorescence with target DNA

| | Target #1 (pX01) | | Target #2 (pX02) | |
|---|---|---|---|---|
| Ba Lysed spores/ | Cycle | End Point | Cycle | End Point |
| 85 uL reaction | Treshold | Flouresence | Treshold | Flouresence |
| 0.0 | 0.0 | 0.2 | 0.0 | 9.7 |
| 400 | 34.22 | 158.2 | 33.66 | 184.5 |
| 4,000 | 30.48 | 351.2 | 30.29 | 299.6 |
| 40,000 | 27.74 | 402.2 | 27.43 | 354.9 |

Each value represents an average of six replicates

What is claimed is:

1. A multiple bead assay system for nucleic acid amplification with an internal control oligonucleotide, the multiple bead assay system comprising a set of reagent beads, wherein the set of reagent beads comprises:
a) a first lyophilized reagent bead comprising:
(i) at least one enzyme for nucleic acid amplification; and
(ii) nucleotides, buffers, salts, or cofactors; and
b) a second lyophilized reagent bead comprising:
i) oligonucleotide primers for specific amplification of the control oligonucleotide; and
ii) primers for specific amplification of at least one analyte nucleic acid sequence.

2. The multiple bead assay system of claim 1, wherein the internal control oligonucleotide is in the first lyophilized reagent bead.

3. The multiple bead assay system of claim 1, wherein the internal control oligonucleotide is in the second lyophilized reagent bead.

4. The multiple bead assay system of claim 3, wherein the second lyophilized reagent bead further comprises probes for detecting the control oligonucleotide and the analyte nucleic acid sequence.

5. The multiple bead assay system of claim 1, further comprising a third lyophilized reagent bead.

6. The multiple bead assay system of claim 5, wherein the third lyophilized reagent bead comprises at least one probe for detection of nucleic acid amplification product.

7. The multiple bead assay system of claim 1, wherein the second lyophilized reagent bead comprises primers for specific amplification of at least two analyte nucleic acid sequences.

8. The multiple bead assay system of claim 7, wherein the second lyophilized reagent bead further comprises probes for detecting the control oligonucleotide and the analyte nucleic acid sequences.

9. The multiple bead assay system of claim 1, wherein the second lyophilized reagent bead comprises primers for specific amplification of at least three analyte nucleic acid sequences.

10. The multiple bead assay system of claim 9, wherein the second lyophilized reagent bead further comprises probes for detecting the control oligonucleotide and the analyte nucleic acid sequences.

11. A multiple bead assay system for nucleic acid amplification, the multiple bead assay system comprising a set of reagent beads, wherein the set of reagent beads comprises:
a) a first lyophilized reagent bead comprising:
(i) at least one enzyme for nucleic acid amplification; and
(ii) nucleotides, buffers, salts, or cofactors; and
b) a second lyophilized reagent bead comprising oligonucleotide primers for amplification of at least one analyte nucleic acid sequence.

12. The multiple bead assay system of claim 11, wherein the second lyophilized reagent bead comprises at least one probe for detection of nucleic acid amplification product.

13. The multiple bead assay system of claim 11, further comprising a third lyophilized reagent bead.

14. The multiple bead assay system of claim 13, wherein the third lyophilized reagent bead comprises a probe for detection of nucleic acid amplification product.

15. The multiple bead assay system of claim 11, wherein the second lyophilized reagent bead comprises primers for amplification of at least two analyte nucleic acid sequences.

16. The multiple bead assay system of claim 15, wherein the second lyophilized reagent bead further comprises probes for detecting the analyte nucleic acid sequences.

17. The multiple bead assay system of claim 1, wherein the second lyophilized reagent bead comprises primers for amplification of at least three analyte nucleic acid sequences.

18. The multiple bead assay system of claim 17, wherein the second lyophilized reagent bead further comprises probes for detecting the analyte nucleic acid sequences.

19. A method for performing a nucleic acid amplification assay with an internal control oligonucleotide, the method comprising the steps of:
a) combining in aqueous solution:
i) a first lyophilized reagent bead that comprises at least one enzyme for nucleic acid amplification, wherein the first lyophilized reagent bead further comprises nucleotides, buffers, salts, or cofactors; and
ii) a second lyophilized reagent bead that comprises oligonucleotide primers for specific amplification of the control oligonucleotide, wherein the second lyophilized reagent bead further comprises primers for specific amplification of at least one analyte nucleic acid sequence, if the analyte nucleic acid sequence is present in the solution; and
b) performing the nucleic acid amplification assay to amplify the internal control oligonucleotide and the at least one analyte nucleic acid sequence, if present.

20. The method of claim 19, wherein the internal control oligonucleotide is in the first lyophilized reagent bead.

21. The method of claim 19, wherein the internal control oligonucleotide is in the second lyophilized reagent bead.

22. The method of claim 19 further comprising the step of detecting the internal control oligonucleotide and the analyte nucleic acid sequence, if present.

23. The method of claim 22, wherein the detecting step comprises detecting a signal from a probe.

24. The method of claim 23, wherein the second lyophilized reagent bead comprises probes for detecting the internal control oligonucleotide and the analyte nucleic acid sequence.

25. The method of claim 19, further comprising the step of combining a third lyophilized reagent bead in the aqueous solution, wherein the third lyophilized reagent bead comprises a probe for detection of nucleic acid amplification product.

26. The method of claim 19, wherein the second lyophilized reagent bead comprises primers for amplification of at least two analyte nucleic acid sequences, and the step of performing the nucleic acid assay comprises amplifying the at least two analyte sequences, if present in the solution.

27. The multiple bead assay system of claim 26, wherein the second lyophilized reagent bead further comprises probes for detecting the internal control oligonucleotide and the analyte nucleic acid sequences, and the method further comprises the step of detecting the internal control oligonucleotide and the analyte nucleic acid sequences, if present.

28. The method of claim 19, wherein the second lyophilized reagent bead comprises primers for amplification of at least three analyte nucleic acid sequences, and the step of performing the nucleic acid assay comprises amplifying the at least three analyte sequences, if present in the solution.

29. The multiple bead assay system of claim 28, wherein the second lyophilized reagent bead further comprises probes for detecting the internal control oligonucleotide and the analyte nucleic acid sequences, and the method further comprises the step of detecting the internal control oligonucleotide and the analyte nucleic acid sequences, if present.
